(19) United States
(12) Patent Application Publication
(10) Pub. No.: US 2014/0276678 A1
(43) Pub. Date: Sep. 18, 2014

(54) SYSTEMS AND DEVICES FOR SHAPING HUMAN CORNEA AND METHODS OF USE THEREOF

(71) Applicants: Michael Berry, Austin, TX (US); Olivia Serdarevic, Goshen, NY (US); Donald F. Heller, Somerset, NJ (US)

(72) Inventors: Michael Berry, Austin, TX (US); Olivia Serdarevic, Goshen, NY (US); Donald F. Heller, Somerset, NJ (US)

(21) Appl. No.: 14/215,772

(22) Filed: Mar. 17, 2014

Related U.S. Application Data
(60) Provisional application No. 61/787,862, filed on Mar. 15, 2013.
(60) Provisional application No. 61/793,867, filed on Mar. 15, 2013.
(60) Provisional application No. 61/793,371, filed on Mar. 15, 2013.
(60) Provisional application No. 61/793,338, filed on Mar. 15, 2013.
(60) Provisional application No. 61/793,624, filed on Mar. 15, 2013.

(51) Int. Cl. A61F 9/008

(52) U.S. Cl. CPC A61F 9/008
USPC 606/5

(57) ABSTRACT

In some embodiments, the instant invention provides for a system for shaping a human cornea of an eye that includes at least the following components: a sapphire applanation window/suction ring (SAWSR) system, where the SAWSR system includes a sapphire applanation window/suction ring (SAWSR), a conical holder, an illuminator, and a temperature control, where the SAWSR system is configured to: (i) be positioned on the eye, (ii) applanate the human cornea of the eye, (iii) generate and position a centration aid, and (iv) maintain temperature control; an optical delivery system, where the optical delivery system includes: (i) a laser, (ii) a fiber delivery holder, and (iii) a laser control subsystem, where the laser control subsystem is configured to display a user interface to: 1) control a power and a temporal waveform of each of the beamlets of light, and 2) irradiate the human cornea of the eye.

### RELATED APPLICATIONS

This application claims the priority of U.S. provisional application Ser. No. 61/787,862, entitled “LASER DEVICES WITH INDIVIDUALLY CONTROLLED LASERS AND/OR LASER BEAMS FOR SHAPING HUMAN CORNEA AND METHODS OF USE THEREOF,” filed Mar. 15, 2013; U.S. provisional application Ser. No. 61/793,867, entitled “MECHANISMS OF MAINTAINING THERMOSTATED TEMPERATURE IN OPERATING LASER DEVICES UTILIZED FOR SHAPING HUMAN CORNEA AND METHODS OF USE THEREOF,” filed Mar. 15, 2013; U.S. provisional application Ser. No. 61/793,371, entitled “AUTOMATIC SUCTION MECHANISMS FOR LASER DEVICES UTILIZED FOR SHAPING HUMAN CORNEA AND METHODS OF USE THEREOF,” filed Mar. 15, 2013; U.S. provisional application Ser. No. 61/793,338, entitled “BEAM SHAPING PATTERNS TO BE UTILIZED WITH LASER DEVICES FOR SHAPING HUMAN CORNEA AND METHODS OF USE THEREOF,” filed Mar. 15, 2013; and U.S. provisional application Ser. No. 61/793,624, entitled “ALIGNMENT MECHANISMS FOR LASER DEVICES UTILIZED FOR SHAPING HUMAN CORNEA AND METHODS OF USE THEREOF, which is incorporated herein by reference in its entirety for all purposes.

### TECHNICAL FIELD

In some embodiments, the instant invention is related to devices, such as laser thermal keratoplasty (LTK), that utilize a laser light to heat corneal tissue for shaping human cornea and methods of use thereof

### BACKGROUND OF INVENTION

In some instances, a human cornea is reshaped for treating a variety of optical defects. The cornea is the transparent front part of the eye that covers the iris, pupil, and anterior chamber. The cornea refracts light, contributing to the majority of the eye's focusing power. Various eye surgery techniques change the shape of the cornea for the purpose of reducing the need for corrective lenses or improving the refractive state of the eye.

### BRIEF SUMMARY OF INVENTION

In some embodiments, the instant invention provides for a system for shaping a human cornea of an eye that includes at least the following components: a sapphire applanation window/suction ring (SAWSR) system, where the SAWSR system includes a sapphire applanation window/suction ring (SAWSR), a conical holder, an illuminator, and a temperature control, where the SAWSR system is configured to: (i) be positioned on the eye, (ii) applanate the human cornea of the eye, (iii) generate and position a centration aid, on the eye, and (iv) maintain temperature control; an optical delivery system, where the optical delivery system at least includes: (i) at least one laser, where the at least one laser is thermally stabilized, (ii) a fiber delivery holder, where the fiber delivery holder is mounted to the SAWSR, where the fiber delivery holder at least includes a plurality of optical fibers, where the at least one laser is optically coupled to the fiber delivery holder, by being one of: 1) optically coupled individually to each fiber, and 2) optically coupled to the fiber delivery holder through a plurality of beamlets of light, where each of the plurality of beamlets of light is optically focused onto a respective proximal end of each optical fiber of the plurality of optical fibers of the fiber delivery holder; and (iii) a laser control subsystem, where the laser control subsystem is configured to display a user interface to: 1) at least independently control a power and a temporal waveform of each of the beamlets of light, and 2) irradiate the human cornea of the eye over a period of time in accordance with at least one set of treatment patterns, where each treatment pattern includes a plurality of treatment areas, where each treatment area corresponds to a respective optical fiber delivering a respective beamlet of the plurality of beamlets of light, and where each treatment area is selected to minimize epithelial modifications, where each treatment area at least includes a shape, where the shape is selected from the group consisting of: rectangular, trapezoidal, elliptical, stadium-shaped, arcuate, overlapping circular, and a combination thereof, and where each treatment area is organized into a pattern, where the pattern is selected from the group consisting of: trigonal, tetragonal, pentagonal, hexagonal, octagonal, annular, and a combination thereof.

In some embodiments, the at least one laser is a continuous wave thulium fiber laser operating at a wavelength in a range between 1.9 and 2.0 μm.

In some embodiments, the at least one laser is a semiconductor diode laser.

In some embodiments, the semiconductor diode laser generates the light having at least one wavelength to correspond to a corneal absorption coefficient that is in a range between 50 and 200 cm⁻¹.

In some embodiments, the system includes a pneumatic syringe, where the pneumatic syringe is configured to provide a suction to applanate the cornea of the eye.

In some embodiments, the optical delivery system further includes at least one of: at least one lens; at least one mirror; where the at least one lens is configured to modulate at least one characteristic of the optical delivery system; and where the at least one mirror is configured to modulate at least one characteristic of the optical delivery system.

In some embodiments, each treatment area is 0.5 mm size in at least one dimension.

In some embodiments, each treatment pattern is selected from the group consisting of: symmetrical and asymmetrical.

In some embodiments, the user interface is configured to: (i) one of: image the eye and import the image from a separate device, (ii) align the SAWSR over the eye, and (iii) import diagnostic data at least from the group consisting of: corneal topography, aberrometry, refraction, and visual acuity.

In some embodiments, the user interface is further configured to display a homing beam, where the homing beam is displayed on the centration reference, and where the centration reference is at least one of: a pupil centroid, a limbus centroid, a coaxially sighted corneal light reflex and a corneal vertex.

In some embodiments, the centration aid includes at least one of the following: (i) an eye image with a fixation light on an optical axis and at an optical infinity, where the eye image is shown on a video display and recorded by a video camera attached to a telescope, (ii) guide circles on the video display that match SAWSR image dimensions, and (iii) a centration aid.

In some embodiments, the SAWSR is automatically mounted on the cornea of the eye using a machine vision and at least one actuator.

In some embodiments, the number of beamlets of light is selected from the group consisting of 4, 8, 16, 24, and 48.

In some embodiments, the instant invention provides for a method for shaping a human cornea of an eye that includes at least the following steps: utilizing a sapphire applanation window/suction ring (SAWSR) system, where the SAWSR system includes a sapphire applanation window/suction ring (SAWSR), a conical holder, an illuminator, and a temperature control, where the SAWSR system is configured to: (i) be positioned on the eye, (ii) applanate the human cornea of the eye, (iii) generate and position a centration aid, on the eye, and (iv) maintain temperature control; utilizing an optical delivery system, where the optical delivery system at least includes: (i) at least one laser, where the at least one laser is thermally stabilized, (ii) a fiber delivery holder, where the fiber delivery holder is mounted to the SAWSR, where the fiber delivery holder includes a plurality of optical fibers, where the at least one laser is optically coupled to the fiber delivery holder, by being one of: 1) optically coupled individually to each fiber, and 2) optically coupled to the fiber delivery holder through a plurality of beamlets of light, where each of the plurality of beamlets of light is optically focused onto a respective proximal end of each optical fiber of the plurality of optical fibers of the fiber delivery holder; and (iii) a laser control subsystem, where the laser control subsystem is configured to display a user interface to: 1) at least independently control a power and a temporal waveform of each of the beamlets of light, and 2) irradiate the human cornea of the eye over a period of time in accordance with at least one set of treatment patterns, where each treatment pattern includes a plurality of treatment areas, where each treatment area corresponds to a respective optical fiber delivering a respective beamlet of the plurality of beamlets of light, and where each treatment area is selected to minimize epithelial modifications, where each treatment area includes a shape, where the shape is selected from the group consisting of: rectangular, trapezoidal, elliptical, stadium-shaped, arcuate, overlapping circular, and a combination thereof, and where each treatment area is organized into a pattern, where the pattern is selected from the group consisting of: trigonal, tetragonal, pentagonal, hexagonal, octagonal, annular, and a combination thereof.

### BRIEF DESCRIPTION OF THE FIGURES

The present invention can be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

FIGS. 1-10 are illustrative depictions of devices and processes related to some embodiments of the present invention.

FIGS. 11-12 are illustrative diagrams related to some embodiments of the present invention.

FIG. 13 is a screenshot of a portion of cornea showing an exemplary condition related to some embodiments of the present invention.

FIGS. 14-16 are illustrative diagrams related to some embodiments of the present invention.

FIG. 17 shows exemplary optical devices related to some embodiments of the present invention.

FIG. 18 is an illustrative diagram related to some embodiments of the present invention.

FIG. 19 is an illustrative flowchart related to some embodiments of the present invention.

FIG. 20 is a screenshot related to some embodiments of the present invention.

In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DETAILED DESCRIPTION OF INVENTION

Among those benefits and improvements that have been disclosed, other objects and advantages of this invention can become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive. Any alterations and further modifications of the inventive feature illustrated herein, and any additional applications of the principles of the invention as illustrated herein, which can normally occur to one skilled in the relevant art and having possession of this disclosure, are to be considered within the scope of the invention.

Throughout the specification, the following terms take the meanings explicitly associated herein, unless the context clearly dictates otherwise. The phrases “In some embodiments” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

In some embodiments, the inventive devices of the instant invention can be utilized to at least reduce and/or alleviate/lessen the symptoms of hyperopia (aka farsightedness) by steepening the central cornea to increase its refractive power and to at least reduce and/or alleviate/lessen the symptoms of presbyopia (aka age-related focus dysfunction) by producing corneal multifocality. In some embodiments, the inventive devices of the instant invention can be utilized to at least reduce and/or alleviate/lessen the symptoms of myopia and astigmatism.

In some embodiments, the instant invention utilizing LTK-type devices can include methods that have at least the following steps. A drop of hydrating solute-free solution is applied to the eye. As shown in FIGS. 1-2, a sapphire applanation window/suction ring (SAWSR), together with its accessories (conical holder and ring illuminator), is positioned over the eye after the eye is hydrated. Then, as shown in FIGS. 3-4, the crosshair reticle on the SAW is used for centration on the pupil centroid. Then, as shown in FIGS. 5-6, suction is applied with a pneumatic syringe (not shown) in order to applanate the cornea with the SAW. The cornea is kept cool and is clinically protected from damage. After, as shown in FIGS. 7-8, a hand piece is docked onto the SAWSR using pre-aligned permanent magnets. The hand piece contains 16 optical fibers that are pre-aligned in a pre-determined treatment (Tx) pattern of, for example, two concentric rings with 8 fibers per ring. Then, the cornea is irradiated by laser light over, for example, a 2.5 second period in which 4 sets of 4 Tx spots/set are delivered through the optical fibers. In some embodiments, each spot is irradiated for 150 milliseconds. During each irradiation, the corneal epithelium is kept cool and undamaged while the anterior corneal stroma is heated to produce extracellular matrix change. As shown in FIGS. 9A-9B, the shape of the cornea is changed. Tx spot opacifications, shown in FIG. 10, fade over a period of a few days to a few weeks, depending on Tx energy density (which is varied to obtain the desired Tx effect).

In some embodiments, the inventive devices of the instant invention can use a continuous wave (cw) thulium fiber laser (TFL) operating at a wavelength of 1.93 μm wavelength together with an optical delivery system (ODS; aka beam distribution system—BDS) to produce beamlets of light that are focused onto the proximal ends of optical fibers that are organized in a fiber delivery holder (FDH; aka fiber optic array—FOA) that generates a Tx pattern.

In some embodiments, the inventive devices of the instant invention can be designed in accordance with a schematic diagram of a laser delivery system shown in FIG. 11. In FIG. 11, semiconductor diode lasers (SDLs) are symbolized as diodes: [symbol] (1101). In FIG. 11, the following terms are used: PAL—pulsed Alexandrite laser (which can be used in some embodiments), PCB—printed circuit board, TE—thermoelectric, and USB—universal serial bus.

In some embodiments, the inventive devices of the instant invention, designed, for example, in accordance with the diagram of FIG. 11, utilize a plurality of lasers such as, but are not limited to, SDLs, which are individually controllable and that are individually coupled to nominal 200 μm core diameter optical fibers that in turn are coupled to the optical fibers in the FDH.

In some embodiments, the inventive devices of the instant invention utilize at least 4 to 16 individually controllable lasers that are individually coupled to respective optical fiber(s) in the FDH. In some embodiments, the inventive devices of the instant invention utilize at least 4 to 24 individually controllable lasers that are individually coupled to respective optical fiber(s) in the FDH. In some embodiments, the inventive devices of the instant invention utilize at least 4 to 48 individually controllable lasers that are individually coupled to respective optical fiber(s) in the FDH. In some embodiments, the inventive devices of the instant invention utilize at least 8 to 16 individually controllable lasers that are individually coupled to respective optical fiber(s) in the FDH. In some embodiments, the inventive devices of the instant invention utilize at least 8 to 24. In some embodiments, the inventive devices of the instant invention utilize an even number of individually controllable lasers that are individually coupled to respective optical fiber(s) in the FDH. Consequently, while the further description in the present invention is in view of SDLs but such description is not limited to only the use of SDLs but similar suitable laser systems can be utilized as well in the manner disclosed herein.

In some embodiments, the inventive devices of the instant invention can utilize at least one laser whose output beam is then split into two or more “beamlets” where each “beamlet” is independently controlled. For example, in some embodiments, the disclosure of the laser systems described in U.S. Pat. No. 8,603,081 is further modified by splitting a single output laser beam from a laser of the U.S. Pat. No. 8,603,081 (106) into multiple beams, all of which are independently controlled (instead of being balanced to be substantially the same in power and duration) to achieve the same function of independently delivering pre-determined energy to each Tx spot. A sample description follows:

A—a laser beam is directed into a beam distribution system,
B—the beam distribution system includes a shutter for providing a correct exposure duration of the laser light, a beamsplitting optical system comprising one or more beamsplitters to produce beamlets, beamlet steering and focusing optics to direct focused beamlet light into optical fibers, a translation stage to move optical fiber arrays into position to received focused beamlet light, a position controller to position the translation stage and
C—beamlet attenuators and/or beamlet modifiers to adjust the amount of focused beamlet light directed into optical fibers; these beamlet attenuators and/or modifiers can be independently controlled to adjust the amount of predetermined beamlet light directed into individual optical fibers.

The specific disclosure of the laser systems described in U.S. Pat. No. 8,603,081 is hereby incorporated by reference herein in its entirety.

Also, in principle, a minimum of 3, or other odd-numbers of, independently controlled lasers (or laser beams) could be used, but this would be more complex than even-numbers of plural lasers because adjustments of vector components of delivered energy would have to be considered. Since the cornea has anisotropic biomechanical properties, adjusting vector components for odd-numbers of plural lasers adds unnecessary complexity. It is much less complex to use inherent corneal symmetries (such as even-numbers of independently controlled lasers and nearly axisymmetric biomechanical properties) for Txs.

In some embodiments, the array of individual lasers is positioned on a common plate that acts as a common heat sink.

In some embodiments, the SDLs are operated at a wavelength between 1.9 and 2.0 μm. In some embodiments, the SDLs are operated at a wavelength of 1.90 μm. In some embodiments, the SDLs are operated at a wavelength of 1.93 μm. In some embodiments, SDLs are operated such as to maintain the absorption coefficient (μₐ) between 50 and 200 cm⁻¹. In some embodiments, SDLs are operated such as to maintain the absorption coefficient (μₐ) at 110 cm⁻¹. In some embodiments, SDLs are operated such as to maintain the absorption coefficient (μₐ) between 100 and 200 cm⁻¹. In some embodiments, SDLs are operated such as to maintain the absorption coefficient GO between 150 and 200 cm⁻¹. In some embodiments, SDLs are operated such as to maintain the absorption coefficient GO between 100 and 150 cm⁻¹. In some embodiments, SDLs are operated such as to maintain the absorption coefficient GO between 50 and 100 cm⁻¹. In some embodiments, SDLs are operated such as to maintain the absorption coefficient GO above 100 cm⁻¹.

In some embodiments, having individually controlled lasers (e.g., SDLs), the inventive devices of the instant invention are designed to individually vary amount and/or duration of light supplied by each laser (e.g., SDL) to their respective optical fiber(s).

In some embodiments, the SDLs are thermally stabilized at a certain wavelength and optionally cooled (if necessary) by thermoelectric (TE) coolers that are thermally coupled to the SDLs using heat sink(s) with high thermal mass(es). In some embodiments, the operational parameters of the SDLs are substantially equivalent to operational parameters of the cw TFL.

In some embodiments, a shutter shown in FIG. 11 is used to change the duration of laser irradiation of the cornea when the SDLs are used in a cw mode continuously. In some embodiments, instead of utilizing the shutter, the inventive devices of the instant invention use the SDLs in a pulsed mode in which either the SDLs are inactive until activated by pulsed electrical current (i.e., “on/off” switching). In some embodiments, instead of utilizing the shutter, the inventive devices of the instant invention use the SDLs in a pulsed mode in which SDLs are in “simmer mode” (active but below the current threshold at which laser action occurs) and are then boosted above threshold by pulsed electrical current. In some embodiments, instead of utilizing the shutter, the inventive devices of the instant invention use the SDLs in a variable pulsed mode in which one or more SDL power outputs have predetermined waveforms including a variable waveform that has at least one of the following variations: “ramping up” the power over the duration of the irradiation, maintaining a constant power over the duration of the irradiation, and controlling a more complicated output of power over the duration of the irradiation.

In some embodiments of the inventive devices of the instant invention, a laser beam from each laser of the plurality of lasers is directly supplied into its corresponding optical fiber and the characteristics of the supplied beam are modulated by the operational characteristics of the laser itself. In some embodiments of the inventive devices of the instant invention, a laser beam from each laser of the plurality of lasers is further passed through at least one optical component (e.g., lenses, mirrors, etc.) that further modulates at least one characteristic of the laser beam before the laser beam reaches its corresponding optical fiber.

In some embodiments, an independent control of pulse durations in each SDL can also permit more versatile treatments to at least reduce and/or alleviate/lessen the symptoms of astigmatism and other Indications for Use (IFUs).

In some embodiments, the inventive devices of the instant invention at least include a microprocessor control board subsystem linked by USB to a laptop computer (or, optionally, to a tablet PC, iPad or smartphone) based User Interface (UI). In some embodiments, the inventive devices of the instant invention can utilize a Light Age Microprocessor Board (MB) with an attached custom-designed Interface Board (IB). In some embodiments, the MB-IB control subsystem controls all of the SDLs, controls the internal shutters (if necessary) and/or any additional interlocks, mediates and/or oversees the firing of the lasers from the footswitch, and/or controls and supervises the DC power from the 150 W power supply to provide electrical power to the SDLs. In addition, in some embodiments, the MB-IB subsystem controls, coordinates, and verifies the calibration. In some embodiments, the inventive devices of the instant invention at least include a counter/enablement subsystem that registers patient treatments (Txs), distinguishes Txs from calibration shots and enables prepaid and/or billed Txs.

In some embodiments, the inventive devices of the instant invention at least include software driven User Interface (UI). In some embodiments, the UI receives inputs from operators through a keyboard, touch screen panel and/or voice recognition software. In some embodiments, the UI not only provides the user settings for the laser system, but also gives password protection for the user, logs and/or archives data, and/or provides technical diagnostics and/or real-time information for operation and maintenance of the system. In some embodiments, the UI uses patient ocular measurements to determine patient treatment requirements, including, but are not limited to, acquisition, tracking and pointing (ATP) of ocular image data for mounting a SAWSR subsystem, and/or for specifying and controlling SDL energy delivery to each optical fiber in the FDH.

In some embodiments, the UI is in a form of a touchscreen control UI that connects to the MB via cable-linked, wireless USB and/or Bluetooth accessory and/or connects to the Internet. In some embodiments, communications include uploading of patient records and/or videos (following compression if necessary) to a network server and/or downloading of software updates and information from the network server. In some embodiments, the inventive devices of the instant invention allow separating the user interface from the main processor, thus the tasks for setting procedure protocols and/or data archiving are isolated from the direct operations of the inventive devices.

In some embodiments, the inventive devices of the instant invention reduce cost and reduce system complexity. In some embodiments, since discrete SDLs can have individual output power monitoring, as well as individual correlated-channel control and monitoring at the distal end of the fiber delivery holder (FDH), the system calibration and resultant optical “dose” delivery to the patient is made more precise and more reproducible by the inventive devices of the instant invention. In some embodiments, the inventive devices of the instant invention allow to direct individual SDL energies to each treatment (Tx) spot, to at least reduce or alleviate/lessen the symptoms of astigmatism and other non-spherical shape changes. In some embodiments, individual SDL energy (and/or timing) can adjust doses in each Tx spot to overcome epithelial thickness variation since such variation may be present pre-Tx and the variation may also change post-Tx, as has been observed for other laser vision correction procedures. In some embodiments, the instant invention utilizes the data that considers epithelial thickness as a function of time post-Tx. In some embodiments, the instant invention utilizes the data that considers SDL energy dosimetry delivered to the corneal stroma (e.g., the principal corneal structure that is modified thermally to produce shape change) can be dependent on epithelial thickness; and thus can compensate for epithelial thickness variations by adjusting laser energy at each Tx spot location. In some embodiments, the instant invention utilizes the data from optical and/or ultrasonic epithelial thickness profiling instruments to obtain epithelial thickness maps.

In some embodiments, the inventive devices of the instant invention utilize direct fiber-to-fiber coupling of each laser that allows reducing the number of mechanical and optical components. In some embodiments, the inventive devices of the instant invention allow for “drop-in” replacement of any SDL in the array.

In some embodiments, once the SAWSR assembly is properly mounted, there are no requirements for acquisition, tracking and pointing (ATP); the magnet-to-magnet docking of the FDH onto the SAWSR provides accurate alignment of the laser Tx pattern onto the cornea. In some embodiments, once the SAWSR assembly is mounted, small patient eye motion does not matter.

Illustrative Examples of Beam Shaping Patterns of the Instant Invention

FIG. 12 shows a pattern of the 16 spots that is utilized in a typical Tx.

In some embodiments, the inventive patterns of the instant invention can be utilized for corneal shaping with treatment conditions that reduce regression due to epithelial modifications and that produce optimal quality of vision. In some embodiments, the inventive patterns of the instant invention allow to select laser irradiation parameters—for example, treatment (Tx) patterns and irradiation distributions—carefully to reduce epithelial modifications that produce regression of Tx effect.

In some embodiments, the Tx patterns can be in the form of rings of Tx spots described above regarding FIG. 12. As shown in FIG. 13, in some case, there are depressions or “dimples” in the Tx spots due to compaction of stromal tissue in the heat-affected zone (HAZ) produced by laser heating. In some cases, epithelial remodeling (e.g., epithelial modification such as epithelial hyperplasia) occurs to “fill in” these depressions. In some embodiments, the inventive patterns of the instant invention and the uses thereof reduce epithelial remodeling, thereby producing more “stable” outcomes. In some embodiments, the inventive patterns of the instant invention minimize surface “irregularity” (i.e., small-scale spatial change in either concavity or convexity) to minimize epithelial modification; the “scale” of spatial change in surface curvature should be greater than ca. 0.5 mm to reduce surface “smoothing” by epithelial modification. In FIG. 13, the Tx spots (with centers identified by arrows) are shown in a porcine cornea histology cross-section. Treated stromal heat-affected zones (HAZs) stain more darkly, are depressed from the anterior surface (top), and are located underneath the epithelial cell layer. Centers of the adjacent Tx spots are 600 μm apart. There is a large “gap” between adjacent Tx spots in which the corneal stroma is untreated. Epithelial cell densities are different within Tx spots compared to between Tx spots; cell locomotion may be linked to cell contact inhibition and other cell signaling phenomena. Post-Tx epithelial modification may partly “fill in” the depressions and may also act to produce a smoother anterior epithelial surface than the very irregular surface evident in FIG. 13.

In some cases, during the inventive treatment (see FIG. 12), the individual Tx spots are ≦0.5 mm “diameter” or “spot size” D (where D is considered to be the full width at half maximum depth—FWHM); the “spot size” increases with increasing Tx energy density in continuous wave (cw) irradiations for a constant irradiation time, but has a maximum value of ca. 0.5 mm. The Tx spots are also discrete so that there are “gaps” between depressions produced in each spot as shown in FIG. 13.

In some cases, the “gaps” between the Tx spots contribute to small-scale spatial changes in surface curvature (and to irregular anterior epithelial shape). In order to reduce regression of the effect due to epithelial modification, the instant invention provides larger “spot sizes” (to exceed the 0.5 mm “scale”) and eliminates or at least sufficiently reduces “gaps” between spots as well as to produces a “smoother” anterior surface.

In some embodiments, the instant invention utilizes elliptical or other non-circular cross-section optical fibers to produce elongated Tx spots and higher laser powers to cover the “footprint” that has the approximate area of two or more circular Tx spots, forming an overlapping circular Tx segment/area. In another embodiment, the instant invention utilizes arcuate and annular or another shape to achieve visual acuity while minimizing corneal surface contour irregularities to minimize corneal epithelial remodeling.

In some embodiments, the instant invention further utilizes in its principles of operation that, for example, stromal lamellae are anisotropic and interwoven in three dimensions in the anterior stroma, and “sutural” lamellae may have significant effects on the anterior cornea surface shape. This means that tissue displacement effects may not be localized. Instead, localized depressions may lead to non-localized displacements elsewhere in the cornea. This non-localized shape change, which may be connected to corneal multifocality produced by the inventive treatment, can be incorporated into determinations performed in some embodiments of the instant invention. Additionally, polarized light microscopy can be used to examine treated spots for increased birefringence (due to compaction of birefringent collagen fibers and lamellae) and to thereby optimize treatment conditions determined using models described in U.S. Pat. No. 8,603,081, whose specific disclosure about those models is hereby incorporated by reference herein in its entirety, to alter the corneal extracellular matrix by, for example, change of corneal hydration state accompanied by proteoglycan conformational change to achieve closer packing of collagen fibrils. A model must be developed that incorporates patient factors (such as age and pre-treatment visual acuity improvement needs) and that incorporates temperature-time histories of thermally mediated processes (such as extracellular matrix changes) that are functions of laser irradiation parameters (such as wavelength, irradiance distribution and irradiation time), tissue composition (which may vary as a function of position within the stroma), reaction and transport kinetics and mechanical loading (such as intraocular pressure and suction pressure).

In some embodiments, the instant invention utilizes overlapping Tx spots such as those illustrated in FIG. 14. FIG. 14 shows a 24-spot Tx pattern. In such pattern, concentric rings are at 1 mm diameter intervals and are centered with respect to the pupillary centroid. In some embodiments, the Tx spots (variable diameter) are located symmetrically and radially on rings at 6.0, 6.8 and 7.6 mm centerline diameters. There are at least the following differences between patterns of FIGS. 12 and 14:

i) Tx spots (of equal diameter with variable spacing between spots on each semimeridian) are overlapped to fill in “gaps” and

ii) additional spots are positioned to form a HAZ that is approximately a “line segment” on each semimeridian.

In some embodiments, the instant invention utilizes variable numbers of Tx spots on each semimeridian in order to provide more effective Txs.

In some embodiments, Tx spots of variable diameter are utilized such as those shown in FIG. 15. It is possible that “tapered” Tx areas (narrower toward the center and wider toward the periphery) may be more effective in optimizing multifocality. FIG. 15 shows a 24-spot Tx pattern with unequal spot sizes. In such pattern, concentric rings are at 1 mm diameter intervals and are centered with respect to the pupillary centroid. In some embodiments, the Tx spots (shown in green; variable diameter) are located symmetrically and radially on rings at 6.0, 6.5 and 7.2 mm centerline diameters.

In some embodiments, the instant invention utilizes other suitable Tx patterns (for example, with only 4 sets of Tx spots, etc.). In some embodiments, the suitable Tx patterns are used in swine eyes and keratometric changes (and also elevation changes) are measured using, for example, a corneal tomography device that provides information on corneal displacements of both anterior and posterior corneal surface points over a large (for example, 10 mm diameter) area of the cornea.

In some embodiments, for the Tx patterns with overlapping Tx spots, the instant invention utilizes coupling of distal optical fiber ends to beam-combining fixtures mounted in the handpiece that is used to deliver laser light. In some embodiments, distal optical fiber ends are ground so that they have flat sides in apposition; one design for three fiber ends is to grind one flat face on each of two fibers together with two flat faces (opposing each other on the middle fiber) on the third fiber so as to combine the flat ends to yield a stadium-like triad.

In FIGS. 12, 14 and 15, all the Tx spots have minimum 6.0 mm centerline ring diameters in order to prevent possible ocular disturbances associated with optical aberrations that extend into the pupil under mesopic illumination conditions. In some embodiments, the instant invention reduces the inner centerline ring diameter to 5.5 mm or even 5.0 mm without causing significant aberrations.

In some embodiments, the instant invention allows for corneal shaping to cause the stromal HAZ to be shaped to yield the best quality of multifocal vision. In some embodiments, the instant invention provides corneal shape change that yields not only excellent distance, intermediate and near visual acuity (hence, multifocality for visual tasks at different object distances—aka simultaneous vision), but also excellent contrast sensitivity, stereoacuity and/or other outcomes that contribute to total quality of vision.

In some embodiments, the inventive patterns of the instant invention utilize treatment (Tx) patterns and irradiation distributions to reduce epithelial modifications that produce regression of Tx effect. For example, FIG. 16 shows Tx rectangular segments/areas oriented along semimeridians spaced at 45° (degrees) intervals. In FIG. 16, concentric rings are at 1 mm diameter intervals and are centered with respect to the pupillary centroid. In some embodiments, the Tx rectangular segments/areas can have variable length and width, and are located symmetrically and radially on semimeridians spaced at 45° intervals.

In FIG. 16, rectangular segments/areas are ca. 1.25 mm long×0.45 mm wide and extend from approximately 5.5 mm ring diameter to 8.0 mm ring diameter. In some embodiments, the inventive patterns of the instant invention utilize different rectangular segment/area sizes (for example, longer or shorter as well as wider or narrower) and locate these segments/areas between different ring diameters. In some embodiments, the device of the instant invention utilizes trapezoidal segments/areas and/or to provide rounded ends to produce a “stadium” or “lozenge” shape. In some embodiments, laser irradiation within each of the Tx areas (for example, rectangular segments/areas such as shown in FIG. 16) is sufficiently nearly uniform (e.g., “flat-top” along both the long and short axes) or can have some other distribution (for example, Gaussian along the short axis and “flat-top” along the long axis). In some embodiments, the resulting heat-affected zones (HAZs) produced by laser irradiation are sufficiently smooth and have small gradients in elevation or depression so to produce corneal shape change without inducing extensive epithelial modifications such as epithelial hyperplasia that “fill in” depressions in the corneal surface.

In some embodiments, outputs from optical fibers can be modified by additional optics in order to produce Tx areal shapes such as those described above. For example, FIG. 17 shows laser “line” generation by a cylindrical lens (left) and a Powell lens (right), one type of aspheric lens (Powell Lens can be bought from, e.g., Altechna; http://www.altechna.com/product_details.php?id=1158). As shown in FIG. 17, the Tx line patterns produced by a cylindrical lens (left) and a Powell lens (right). The cylindrical lens yields a non-uniform (Gaussian) irradiance distribution while the Powell lens yields a uniform irradiance distribution within the line (actually rectangle) segment/area.

In some embodiments, Powell lenses can be manufactured with different “fan angles” to produce different “line” lengths; “line” lengths can also be increased by increasing the spacing between the flat (exit) face of the lens from the substrate. In some embodiments, the Powell microlenses in accordance with the instant invention would have to be manufactured using a transparent material (such as low OH silica) and these lenses would have to be mounted in an assembly that spaces the lenses correctly in apposition with optical fibers so as to produce a Tx pattern similar to that shown in FIG. 16.

In some embodiments, the inventive patterns of the instant invention utilize the device of the instant invention, such as LTK, to at least reduce and/or alleviate/lessen the symptoms of hyperopia (aka farsightedness) by steepening the central cornea to increase its refractive power.

In some embodiments, the double spot patterns along semimeridians of FIG. 12 can be replaced by rectangular segment/area or stadium-shaped Tx regions as shown in FIG. 16. This pattern of radial segments/areas produces central corneal steepening which can be used to at least reduce and/or alleviate/lessen the symptoms of hyperopia. In some embodiments, this Tx pattern produces “tightening” of corneal tissue by thermal modification.

In some embodiments, to at least reduce and/or alleviate/lessen the symptoms of astigmatism using the Tx patterns shown in FIG. 16, the radial rectangular segments/areas may be treated at different Tx energy densities and/or rotated to be centered on semimerdians different from those shown in the FIG. 16.

In some embodiments, since the radial pattern produces “tightening” that leads to the opposite effect of the RK “cutting” procedure, the instant invention utilizes other patterns to produce opposite effects—viz., hexagonal Tx patterns (e.g., HLTK) produces the opposite effect (central corneal flattening to at least reduce and/or alleviate/lessen the symptoms of myopia) of hexagonal keratotomy (HK) used to at least reduce and/or alleviate/lessen the symptoms of hyperopia, and astigmatic Tx patterns (e.g., ALTK) should produce the opposite effect of astigmatic keratotomy (AK) using either arcuate or transverse cuts. In some embodiments, HLTK should not produce HK complications such as globe rupture since HLTK does not damage or weaken the cornea.

In some embodiments, the devices of the instant invention utilize some other Tx patterns besides hexagonal Tx patterns. For example, FIG. 18 shows an octagonal Tx pattern (e.g., OLTK). In FIG. 18, the octagonal Tx pattern is used to at least reduce and/or alleviate/lessen the symptoms of myopia, including concentric rings that are at 1 mm diameter intervals and are centered with respect to the pupil centroid. Tx transverse rectangular segments/areas have variable length and width, and are located symmetrically on semimeridians spaced at 45° intervals. In some embodiments, to at least reduce and/or alleviate/lessen the symptoms of astigmatism, in addition to myopia and presbyopia, these transverse rectangular segments/areas are treated at different Tx energy densities and/or are rotated to be centered on semimeridians different from those shown in FIG. 18.

In some embodiments, the inventive patterns of the instant invention utilize yet some other LTK Tx patterns such as trigonal LTK, tetragonal LTK, pentagonal LTK, etc. In some embodiments, the octagonal LTK yields an axisymmetric pattern with high versatility. In some embodiments, the devices of the instant invention utilize arcuate patterns in which arc lengths are circumferential or non-circumferential in order to obtain “tailored” keratoplasty effects. In some embodiments, the inventive patterns of the instant invention achieve equivalent visual acuity minimizing corneal surface contour irregularities to minimize corneal epithelial remodeling.

In some embodiments, the inventive patterns of the instant invention utilize the astigmatic Tx patterns (e.g., ALTK) for at least reducing or alleviating/lessening the symptoms of astigmatism that involve a subset of Tx areas or variable Tx energies within each Tx area. For example, for correction of eyes without a spherical error but “with-the-rule” astigmatism (in which the cornea is steeper along the vertical—90°-270°—meridian), the two transverse rectangular segments/areas centered at 90° and 270° in FIG. 18 could be treated to produce some flattening along the vertical meridian. In some embodiments, the magnitude of the astigmatic Tx (e.g., ALTK) effect depends on the length of the transverse rectangular (or arcuate) segments/areas, their location (i.e., polar coordinates on polar maps such as are used in FIGS. 12, 16 and 18), and patient age. In some embodiments, the magnitude of effect depends on the Tx energy density (which also affects the width and depth of the transverse rectangular volumes that are treated within each segment/area). In some embodiments, the magnitude of reducing or alleviating/lessening the symptoms of myopia is also influenced by the same variables (Tx energy density; treated volumes—length, width and depth; patient age).

In some embodiments, the symmetrical Tx patterns described herein above are directed to at least reduce and/or alleviate/lessen the symptoms of spherical refractive errors (myopia and hyperopia) and regular (symmetrical) astigmatism. In some embodiments, irregular astigmatism and/or other irregular shape distortions such as keratoconus can also be at least reduced or alleviated/lessened by using the asymmetrical Tx patterns.

In some embodiments, the inventive patterns of the instant invention utilize, a laser producing light at a wavelength of 1.93 μm leading to an absorption coefficient of ca. 110 cm−1 in corneal tissue. In some embodiments, the inventive patterns of the instant invention utilize some other laser wavelengths that have different absorption coefficients that affect the depth of the heat affected zone (HAZ) which in turn affect the magnitude of corneal shape change.

In some embodiments, HAZ dimensions depend, in general, on many factors such as laser wavelength, laser duration, laser irradiance distribution, corneal temperature, epithelial thickness, thermal conduction into a heat sink such as a sapphire window, etc. In some embodiments, the inventive patterns of the instant invention use the minimum amount of total laser energy needed to achieve a predetermined corneal shape change in order to reduce potential collateral damage.

In some embodiments, the inventive patterns of the instant invention allow to mount the SAWSR accurately with respect to the angular orientation of the Tx pattern in order to allow for sequential multiple Txs over time. For example, a patient may have a primary Tx that is followed by a secondary Tx at a later time. For greater effectiveness and predictability, the primary and secondary Tx patterns should not overlap; for example, if the primary Tx pattern includes Txs along the 0°-180° and 90°-270° meridians, the secondary Tx pattern may be best oriented with Txs along the 45°-225° and 135°-315° meridians.

Illustrative Examples of Alignment Mechanisms of the Instant Invention

In some embodiments, the instant invention allows to minimize SAWSR mounting times by displaying a “homing beam” on a monitor that a physician can utilize to mount the SAWSR within a sufficiently short time period and with improved ease and accuracy. In some embodiments, the instant invention allows to minimize SAWSR mounting times by utilizing machine vision plus actuators (such as motion control devices) on the SAWSR control to assist the physician to mount the SAWSR within the sufficiently short time period.

In some embodiments, the inventive devices of the instant invention at least include a software driven User Interface (UI). In some embodiments, the UI receives inputs from operators through a keyboard, touch screen panel and/or voice recognition software. In some embodiments, the UI not only provides the user settings for the laser system, but also gives password protection for the user, log and/or archive data, and/or provide technical diagnostics and/or real-time information for operation and maintenance of the system. In some embodiments, the UI uses patient ocular measurements to determine patient treatment requirements, including, but are not limited to, acquisition, tracking and pointing (ATP) of ocular image data for mounting a SAWSR subsystem, and/or for specifying and controlling energy delivery to each optical fiber in a fiber delivery holder (FDH; aka fiber optic array—FOA) that generates Tx pattern.

In some embodiments, the UI is in a form of a touchscreen control UI that connects to the microprocessor board (MB) via cable-linked, wireless USB and/or Bluetooth accessory and/or connects to the Internet. In some embodiments, communications include uploading of patient records and/or videos (following compression if necessary) to a network server and/or downloading of software updates and information from the network server. In some embodiments, the inventive devices of the instant invention allow separating the user interface from the main processor, thus the tasks for setting procedure protocols and/or data archiving are isolated from the direct operations of the inventive devices.

In some embodiments, the inventive devices of the instant invention utilize direct fiber-to-fiber coupling of each laser that allows reducing the number of mechanical and optical components. In some embodiments, the inventive devices of the instant invention allow for “drop-in” replacement of any SDL in the array.

In some embodiments, the inventive devices of the instant invention are operated in steps shown in FIG. 19. The patient initially views a fixation light that is located on the optical axis defined by a telescope line-of-view displayed on the center of a monitor of the inventive devices (which is pre-aligned). In some embodiments, the fixation can be approximate; it is only necessary for the patient to look in the correct direction (i.e., along the optical axis). In some embodiments, the ATP steps for centration, angulation and normal incidence viewing leading to a “homing beam” superimposed on the monitor compensate for small displacements of the eye view from the optical axis.

In step 1 of FIG. 19, the eye image can be a real-time monitor display (on a screen of a computer portable device such as iPad3, a machine vision display, etc.) and, optionally, an imported image from a separate device; and diagnostic data can be imported from corneal topography, aberrometry, refraction, visual acuity and/or other measurements. In some embodiments, the separate imaging device is a camera that records the imagery of the eye.

In some embodiments, the inventive systems of the instant invention allow for monocular viewing with, for example, the iPad3 by using a telescope finder scope (such as, but is not limited to, the Orion Telescope black 6×30 right-angle correct-image finder scope which provides 6× magnification and has a 30 mm diameter objective lens with 7° field-of-view). In some embodiments, the suitable finder scope is mounted onto the iPad3 on the optical axis of the iPad3's CMOS camera. In some embodiments, a fixation light is also built into the suitable finder scope housing so that the patient eye fixates along the optical axis of the finder scope/camera. In some embodiments, the suitable finder scope is pre-aligned along the optical axis which then serves as a reference for fixation and for mounting the SAWSR assembly so that the optical axis is at normal incidence (i.e., perpendicular) to the sapphire applanation window (SAW). In some embodiments, the Parallax error (that could occur because the sapphire window plane is not the same as the pupillary “plane”) is eliminated by utilizing the monocular viewing, described above, plus the normal incidence geometry. In some embodiments, the inventive systems of the instant invention further include a double reticle and/or a level sensor that are used to verify/confirm the normal incidence viewing. In some embodiments, an equivalent centering system to the double reticle is used to verify/confirm the normal incidence viewing.

In step 2 of FIG. 19, treatment (Tx) parameters are computed from diagnostic data; the inventive devices' characteristics (Tx power and duration in each location) are adjusted automatically (using a Tx nomogram) to provide correct Tx parameters.

In step 3 of FIG. 19, for centration, the pupil edge is found in real-time at 4 or more semimeridans (for example, at 0°, 90°, 180° and 270°); the pupil centroid is the intersection of linear connectors between opposing semimeridians (for example, 0° and 180°). In some embodiments, the pupil centroid is a candidate centration reference onto which a “homing beam” can be projected on a monitor. In some embodiments, in step 3, other choices for centration reference can include the limbus center and the corneal vertex. In some embodiments, in step 3, other reference “markers” can be used for angulation such as, but are not limited to: iris patterns and scleral blood vessels. In some embodiments, angulation accuracy is necessary to treat astigmatism. In some embodiments, the instant invention uses reference “markers” obtained in the supine position since ocular cyclotorsion occurs when a patient changes position from sitting upright to lying supine. In some embodiments, the inventive systems of the instant invention utilize pupillometry with edge detection which incorporates the following steps:

A—Video recording of the image of an eye, including the pupil and the limbus,
B—Application of an edge detection algorithm (such as the Canny edge detector) to locate pupil edges at a predetermined number of semimeridians (for example, at each integral semimeridian from 0° to 359°),
C—Fitting of an ellipse to the array of edges and
D—Location of the center point of the ellipse which is the pupil centroid.
The same procedure can be used to locate the limbus centroid by substituting the limbal edges for pupil edges in step B above.

In step 4 of FIG. 19, to aid the physician in mounting the SAWSR, the instant invention adds a “homing beam” (and angular markers in the case of at least reducing or alleviating/lessening the symptoms of astigmatism) to the monitor display. In some embodiments, the “homing beam” is displayed on the centration reference (such as the pupil centroid). In some embodiments, the reticle center (part of the SAWSR assembly) can be superimposed on the “homing beam” as viewed on the display. In some embodiments, angular markers on the SAWSR can be superimposed on angular markers shown on the display.

In step 5 of FIG. 19, in some embodiments, the physician can mount the SAWSR assembly on the eye. In step 5 of FIG. 19, in some embodiments, machine vision is used to automate the mechanical placement of the SAWSR assembly on the eye. When the SAWSR is properly mounted (with respect to centration, angulation and normal incidence—the latter verified by superposition of double reticle crossmarks or circles in order to avoid parallax error; as an alternative, an electronic level sensor can be used to verify that the SAW is at normal incidence to the optical axis), suction is applied. If the SAWSR is not properly mounted, the suction can be released and the mounting steps are repeated.

In step 6 of FIG. 19, in some embodiments, the fiber delivery holder (FDH) is docked onto the mounted SAWSR; a set of permanent magnets aligns the FDH accurately with respect to the SAWSR. In some embodiments, the FDH is docked manually by the physician. In some embodiments, the FDH's docking is automated. Once docked, the laser treatment (Tx) is performed. In some embodiments, Tx is initiated manually by the physician. In some embodiments, Tx is automatically initiated.

In step 7 of FIG. 19, in some embodiments, following Tx, suction is released and the SAWSR and FDH are removed. In some embodiments, the step 7 is performed manually. In some embodiments, the step 7 is automatically performed.

In some embodiments, the inventive devices/systems of the instant invention allow to fully automate the entire procedure. In some embodiments, the inventive devices of the instant invention utilize machine vision and pattern recognition for ATP of the centration, angulation and normal incidence references. In some embodiments, the inventive devices of the instant invention that utilize the SAWSR assembly should “lock onto” the “homing beam” target and be mounted directly on target.

In some embodiments, once the SAWSR assembly is properly mounted, there are no requirements for ATP; the magnet-to-magnet docking of the FDH onto the SAWSR provides accurate alignment of the laser Tx pattern onto the cornea. In some embodiments, once the SAWSR assembly is mounted, small patient eye motion does not matter.

In some embodiments, the inventive alignment mechanisms/devices of the instant invention are designed to achieve centration for the specific treatment (Tx) patterns to obtain maximum and predictable effectiveness of the inventive procedures utilizing the inventive devices of the instant invention. In some embodiments, the inventive devices of the instant invention utilize at least one of the following centration locations:

i) the pupil centroid (PC),

ii) the corneal vertex (CV),

iii) any other suitable location such as the coaxially sighted corneal light reflex (CSCLR) for patients with significant Angle Kappa.

In some embodiments, the inventive devices of the instant invention allow to mount them, such as a SAWSR, accurately and quickly, without repeated trauma to the cornea caused by multiple mounting attempts and/or excessive mounting adjustments. FIG. 20 shows an eye with the CSCLR (first Purkinje image; marked by a white cross) as the centration reference. The pupil centroid (under photopic illumination conditions; marked by a green cross that nearly overlaps the white cross) was displaced by X=0.145 mm, Y=0.021 mm from the CSCLR. The figure also shows computer-generated edge finding circles (yellow—limbus, green—pupil). The image of FIG. 20 was taken using a SensoMotoric Instruments (SMI) AcuTarget instrument.

In some embodiments, the inventive devices of the instant invention allow to display a “homing beam” on the eye image on the pupil centroid (and/or some other centration reference) to allow the physician to “home in” on his/her mounting target. In some embodiments, it is not necessary to display the pupil (or limbus) edges as shown in FIG. 20. In some examples, a small number of pupil edge points (perhaps only 4 points at 0°, 90°, 180° and) 270° may be sufficient to acquire in order to calculate the pupil centroid. In some embodiments, the “homing beam” on the pupil centroid may be a flashing red light or another very noticeable target.

In some embodiments, the inventive devices of the instant invention additionally utilize at least the following additional centration aids, but are not limited to:

1—a video camera and display with telescope together with a fixation light on the optical axis and at optical infinity,

2—guide circles on the video display that match SAWSR image dimensions, and

3—a double reticle (one on or near the plane of the proximal sapphire window face and the other at least 1 mm apart on the distal sapphire window face) to prevent parallax errors—the spacing between reticles should be as large as possible but should not exceed the depth of field of the telescope optics.

In some embodiments, the inventive devices of the instant invention allow to mount the SAWSR accurately with respect to the angular orientation of the Tx pattern in order to allow for sequential multiple Txs over time. For example, a patient may have a primary Tx that is followed by a secondary Tx at a later time. In some embodiments, the primary and secondary Tx patterns do not overlap; for example, if the primary Tx pattern includes Txs along the 0°-180° and 90°-270° meridians, the secondary Tx pattern may be oriented with Txs along the 45°-225° and 135°-315° meridians.

In some embodiments, accurate angulation is even more important in the context of at least reducing or alleviating/lessening the symptoms of astigmatism. In some embodiments, the instant invention accounts for one or more complicating factors such as cyclorotation of the eye that occurs when a patient lies down. In some embodiments, the inventive devices of the instant invention utilize iris registration to define the angular orientation in terms of fixed marks on the iris. In some embodiments, one or more secondary “homing beams” (in addition to the primary “homing beam” on the PC or other centration reference) may be included on the video display to aid the physician in mounting the SAWSR accurately with respect to both centration and angulation.

In some embodiments, the inventive devices of the instant invention measure the location of the PC, the CV, iris markers, etc. using diagnostic devices such as aberrometers or corneal topographers. In some embodiments, centration and angulation data then is transferred by software from diagnostic devices to some of the inventive devices of the instant invention Tx device for use during Txs.

Illustrative Examples of Automatic Suction Mechanisms of the Instant Invention

In some embodiments, the suction is automated. In some embodiments, the automatic mounting of the SAWSR assembly can include optical detection of the meniscus edge formed by fluid located between the eye and the SAW; when the meniscus spreads sufficiently, an electronic control can start a predetermined amount of suction (such as 30 cm Hg pressure differential). In some embodiments, the inventive systems of the instant invention can utilize the edge-finding of the meniscus edge by the same or similar suitable type of procedure utilized in pupillometry as described above for pupillometry with edge finding.

In some embodiments, the inventive systems of the instant invention can utilize any other suitable systems/devices that optically detect/measure the meniscus without adding complexity and/or significantly increase (e.g., double time) of the inventing Tx methods.

In some embodiments, if the SAWSR is not properly mounted, the suction can be released and the mounting steps are repeated.

Illustrative Examples of Mechanisms of Maintaining Thermostated Temperature in the Inventive Devices of the Instant Invention

In some embodiments, the inventive devices/systems of the instant invention maintain temperature control (at a predetermined thermostated temperature) of the SAW to improve accuracy and/or predictability of LTK treatments (Txs). Typically, there is considerable patient-to-patient variability in ocular surface temperature. Since the inventive treatments depend, at least in part, upon the temperature-time history of laser heating, variations in an initial ocular (e.g., anterior corneal) temperature can alter Tx effects.

In some embodiments, the inventive devices/systems of the instant invention maintain temperature control (at a predetermined thermostated temperature) of the SAW by measuring, continuously and/or periodically, ocular surface temperature using one or more suitable techniques/devices such as noncontact radiometry.

In some embodiments, the inventive devices/systems of the instant invention maintain temperature control (at a predetermined thermostated temperature) of the SAW by also incorporating measurements of variations in room temperature (typically, the room temperature varies from clinic to clinic and/or within a clinic from time to time).

In some embodiments, the inventive devices/systems of the instant invention utilize a feedback loop mechanism by, continuously or periodically, collecting, temperature measurements of at least one of: ocular surface temperature, SAW temperature, and room temperature, —and based on the obtained measurement(s) adjusting SAW's temperature by, for example, performing at least one of the following actions, but is not limited to: blowing hot air, resistive heating of the SAW by, for example, using polyimide resistive heating tape that is in thermal contact with the SAW, and other similarly suitable methods.

In some embodiments, the instant invention provides for a system for shaping a human cornea of an eye that includes at least the following components: a sapphire applanation window/suction ring (SAWSR) system, where the SAWSR system includes a sapphire applanation window/suction ring (SAWSR), a conical holder, an illuminator, and a temperature control, where the SAWSR system is configured to: (i) be positioned on the eye, (ii) applanate the human cornea of the eye, (iii) generate and position a centration aid, on the eye, and (iv) maintain temperature control; an optical delivery system, where the optical delivery system at least includes: (i) at least one laser, where the at least one laser is thermally stabilized, (ii) a fiber delivery holder, where the fiber delivery holder is mounted to the SAWSR, where the fiber delivery holder at least includes a plurality of optical fibers, where the at least one laser is optically coupled to the fiber delivery holder, by being one of: 1) optically coupled individually to each fiber, and 2) optically coupled to the fiber delivery holder through a plurality of beamlets of light, where each of the plurality of beamlets of light is optically focused onto a respective proximal end of each optical fiber of the plurality of optical fibers of the fiber delivery holder; and (iii) a laser control subsystem, where the laser control subsystem is configured to display a user interface to: 1) at least independently control a power and a temporal waveform of each of the beamlets of light, and 2) irradiate the human cornea of the eye over a period of time in accordance with at least one set of treatment patterns, where each treatment pattern includes a plurality of treatment areas, where each treatment area corresponds to a respective optical fiber delivering a respective beamlet of the plurality of beamlets of light, and where each treatment area is selected to minimize epithelial modifications, where each treatment area at least includes a shape, where the shape is selected from the group consisting of: rectangular, trapezoidal, elliptical, stadium-shaped, arcuate, overlapping circular, and a combination thereof, and where each treatment area is organized into a pattern, where the pattern is selected from the group consisting of: trigonal, tetragonal, pentagonal, hexagonal, octagonal, annular, and a combination thereof.

In some embodiments, the at least one laser is a continuous wave thulium fiber laser operating at a wavelength in a range between 1.9 and 2.0 μm.

In some embodiments, the at least one laser is a semiconductor diode laser.

In some embodiments, the semiconductor diode laser generates the light having at least one wavelength to correspond to a corneal absorption coefficient that is in a range between 50 and 200 cm⁻¹.

In some embodiments, the system includes a pneumatic syringe, where the pneumatic syringe is configured to provide a suction to applanate the cornea of the eye.

In some embodiments, the optical delivery system further includes at least one of: at least one lens; at least one mirror; where the at least one lens is configured to modulate at least one characteristic of the optical delivery system; and where the at least one mirror is configured to modulate at least one characteristic of the optical delivery system.

In some embodiments, each treatment area is 0.5 mm size in at least one dimension.

In some embodiments, each treatment pattern is selected from the group consisting of: symmetrical and asymmetrical.

In some embodiments, the user interface is configured to: (i) one of: image the eye and import the image from a separate device, (ii) align the SAWSR over the eye, and (iii) import diagnostic data at least from the group consisting of: corneal topography, aberrometry, refraction, and visual acuity.

In some embodiments, the user interface is further configured to display a homing beam, where the homing beam is displayed on the centration reference, and where the centration reference is at least one of: a pupil centroid, a limbus centroid, a coaxially sighted corneal light reflex and a corneal vertex.

In some embodiments, the centration aid includes at least one of the following: (i) an eye image with a fixation light on an optical axis and at an optical infinity, where the eye image is shown on a video display and recorded by a video camera attached to a telescope, (ii) guide circles on the video display that match SAWSR image dimensions, and (iii) a centration aid.

In some embodiments, the SAWSR is automatically mounted on the cornea of the eye using a machine vision and at least one actuator.

In some embodiments, the number of beamlets of light is selected from the group consisting of 4, 8, 16, 24, and 48.

In some embodiments, the instant invention provides for a method for shaping a human cornea of an eye that includes at least the following steps: utilizing a sapphire applanation window/suction ring (SAWSR) system, where the SAWSR system includes a sapphire applanation window/suction ring (SAWSR), a conical holder, an illuminator, and a temperature control, where the SAWSR system is configured to: (i) be positioned on the eye, (ii) applanate the human cornea of the eye, (iii) generate and position a centration aid, on the eye, and (iv) maintain temperature control; utilizing an optical delivery system, where the optical delivery system includes: (i) at least one laser, where the at least one laser is thermally stabilized, (ii) a fiber delivery holder, where the fiber delivery holder is mounted to the SAWSR, where the fiber delivery holder includes a plurality of optical fibers, where the at least one laser is optically coupled to the fiber delivery holder, by being one of: 1) optically coupled individually to each fiber, and 2) optically coupled to the fiber delivery holder through a plurality of beamlets of light, where each of the plurality of beamlets of light is optically focused onto a respective proximal end of each optical fiber of the plurality of optical fibers of the fiber delivery holder; and (iii) a laser control subsystem, where the laser control subsystem is configured to display a user interface to: 1) at least independently control a power and a temporal waveform of each of the beamlets of light, and 2) irradiate the human cornea of the eye over a period of time in accordance with at least one set of treatment patterns, where each treatment pattern includes a plurality of treatment areas, where each treatment area corresponds to a respective optical fiber delivering a respective beamlet of the plurality of beamlets of light, and where each treatment area is selected to minimize epithelial modifications, where each treatment area includes a shape, where the shape is selected from the group consisting of: rectangular, trapezoidal, elliptical, stadium-shaped, arcuate, overlapping circular, and a combination thereof, and where each treatment area is organized into a pattern, where the pattern is selected from the group consisting of: trigonal, tetragonal, pentagonal, hexagonal, octagonal, annular, and a combination thereof

While a number of embodiments of the present invention have been described, it is understood that these embodiments are illustrative only, and not restrictive, and that many modifications may become apparent to those of ordinary skill in the art.

What is claimed is:

1. A system for shaping a human cornea of an eye comprising:
a sapphire applanation window/suction ring (SAWSR) system,
wherein the SAWSR system comprises a sapphire applanation window/suction ring (SAWSR), a conical holder, an illuminator, and a temperature control,
wherein the SAWSR system is configured to:
(i) be positioned on the eye,
(ii) applanate the human cornea of the eye,
(iii) generate and position a centration aid, on the eye, and
(iv) maintain temperature control;
an optical delivery system,
wherein the optical delivery system comprises:
(i) at least one laser,
wherein the at least one laser is thermally stabilized,
(ii) a fiber delivery holder,
wherein the fiber delivery holder is mounted to the SAWSR,
wherein the fiber delivery holder comprises a plurality of optical fibers,
wherein the at least one laser is optically coupled to the fiber delivery holder, by being one of:
1) optically coupled individually to each fiber, and
2) optically coupled to the fiber delivery holder through a plurality of beamlets of light, wherein each of the plurality of beamlets of light is optically focused onto a respective proximal end of each optical fiber of the plurality of optical fibers of the fiber delivery holder; and
(iii) a laser control subsystem,
wherein the laser control subsystem is configured to display a user interface to:
1) at least independently control a power and a temporal waveform of each of the beamlets of light, and
2) irradiate the human cornea of the eye over a period of time in accordance with at least one set of treatment patterns, wherein each treatment pattern comprises a plurality of treatment areas,
wherein each treatment area corresponds to a respective optical fiber delivering a respective beamlet of the plurality of beamlets of light, and
wherein each treatment area is selected to minimize epithelial modifications,
wherein each treatment area comprises a shape,
wherein the shape is selected from the group consisting of: rectangular, trapezoidal, elliptical, stadium-shaped, arcuate, overlapping circular, and a combination thereof, and
wherein each treatment area is organized into a pattern,
wherein the pattern is selected from the group consisting of: trigonal, tetragonal, pentagonal, hexagonal, octagonal, annular, and a combination thereof.

2. The system of claim 1, wherein the at least one laser is a continuous wave thulium fiber laser operating at a wavelength in a range between 1.9 and 2.0 μm.

3. The system of claim 1, wherein the at least one laser is a semiconductor diode laser.

4. The system of claim 3, wherein the semiconductor diode laser generates the light having at least one wavelength to correspond to a corneal absorption coefficient that is in a range between 50 and 200 cm⁻¹.

5. The system of claim 1, further comprising a pneumatic syringe, wherein the pneumatic syringe is configured to provide a suction to applanate the cornea of the eye.

6. The system of claim 1, wherein the optical delivery system further comprises at least one of:
at least one lens;
at least one mirror;
wherein the at least one lens is configured to modulate at least one characteristic of the optical delivery system; and
wherein the at least one mirror is configured to modulate at least one characteristic of the optical delivery system.

7. The system of claim 1, wherein each treatment area is 0.5 mm size in at least one dimension.

8. The system of claim 1, wherein each treatment pattern is selected from the group consisting of: symmetrical and asymmetrical.

9. The system of claim 1, wherein the user interface is configured to:
(i) one of: image the eye and import the image from a separate device,
(ii) align the SAWSR over the eye, and
(iii) import diagnostic data at least from the group consisting of: corneal topography, aberrometry, refraction, and visual acuity.

10. The system of claim 9, wherein the user interface is further configured to display a homing beam,
wherein the homing beam is displayed on the centration reference, and
wherein the centration reference is at least one of: a pupil centroid, a limbus centroid, a coaxially sighted corneal light reflex and a corneal vertex.

11. The system of claim 1,
wherein the centration aid comprises at least one of the following:
(i) an eye image with a fixation light on an optical axis and at an optical infinity, wherein the eye image is shown on a video display and recorded by a video camera attached to a telescope,
(ii) guide circles on the video display that match SAWSR image dimensions, and
(iii) a centration aid.

12. The system of claim 1, wherein the SAWSR is automatically mounted on the cornea of the eye using a machine vision and at least one actuator.

13. The system of claim 1, wherein the number of beamlets of light is selected from the group consisting of 4, 8, 16, 24, and 48.
